(19) United States
(12) Patent Application Publication
(10) Pub. No.: US 2007/0073248 A1
(43) Pub. Date: Mar. 29, 2007

(54) TROCAR-CANNULA COMPLEX, CANNULA AND METHOD FOR DELIVERING BIOLOGICALLY ACTIVE AGENTS DURING MINIMALLY INVASIVE SURGERY

(75) Inventor: Stephen P. Moenning, Punta Gorda, FL (US)

Correspondence Address: WOOD, HERRON & EVANS, LLP, 2700 CAREW TOWER, 441 VINE STREET, CINCINNATI, OH, 45202, US

(73) Assignee: RxTrocar, Ltd., Punta Gorda, FL (US)

(21) Appl. No.: 11/464,893

(22) Filed: Aug. 16, 2006

Related U.S. Application Data
(63) Continuation of application No. PCT/US05/07678, filed on Mar. 8, 2005.
(60) Provisional application No. 60/552,048, filed on Mar. 10, 2004.

(51) Int. Cl. A61M 25/00

(52) U.S. Cl. 604/264

(57) ABSTRACT

A trocar-cannula complex for use in minimally invasive surgical procedures performed through a port site of a patient and in the delivery of biologically active agents to the patient includes a trocar and a cannula. The cannula includes a tubular structure with a central lumen receiving the trocar and an outer surface adapted to interface with tissue at the port site. A first delivery mechanism is associated with the cannula for delivering a first biologically active agent to a patient and a second delivery mechanism is associated with the cannula for delivery of a second biologically active agent to the patient. Various other manners of delivering the agents are also disclosed.

This application is a continuation of PCT Serial No. PCT/US2005/007678 filed Mar. 8, 2005 which claims the priority of U.S. Provisional Application No. 60/552,048, filed Mar. 10, 2004, the disclosures of which are fully incorporated by reference herein.

### FIELD OF THE INVENTION

This invention generally relates to cannulas and, more specifically, to cannulas used during minimally invasive surgery for allowing the introduction of instruments, such as laparoscopic tools, during surgical procedures and for the delivery of biologically active agents to the port site of the patient.

### BACKGROUND OF THE INVENTION

Minimally invasive surgery is a popular alternative to more traditional surgery. This is due to the fact that minimally invasive surgery generally results in less pain and shorter hospital stays for the patient. Also, the cost of performing a surgical procedure through minimally invasive techniques can be substantially less than more traditional surgical approaches.

Minimally invasive surgical techniques require access into the body of a patient through a small working channel of an apparatus known as a trocar-cannula complex. A relatively small access incision is made in the patient at the appropriate location on the patient to receive the trocar-cannula complex. When the trocar-cannula complex is combined with long, narrow instruments, the resulting assembly allows a surgeon to work inside the body through the small access incision or port site. This approach has resulted in the aforementioned clinical advantages and extensive health care cost savings.

Traditionally, the trocar-cannula complex has been configured with three parts. The first part is the top portion and is referred to in the medical industry as the hub. The hub defines the entrance to the trocar-cannula complex and also includes various seals and air insufflation components. The second part is the trocar, which is a long, narrow blade extendable through an inner cannula to allow smooth penetration into the body of the patient through the tissue layers. The third portion is an outer cannula which is a tubular member of the complex adapted to pass into the body cavity. The outer cannula provides an interface between the patient's tissue at the access incision or port site and the trocar assembly.

Minimally invasive surgery has grown in popularity in recent years and many new types of trocar-cannula products have been proposed and introduced to address different surgical needs and procedures. The various trocar-cannula complexes include reusable and disposable cannulas and trocars, as well as hybrid varieties that comprise combinations of reusable and disposable components of the trocar-cannula complexes. A complex which is a combination of reusable and disposable components is known as a resposable device. Such devices continue to improve surgical outcomes and economics.

Animal studies on cancer treatments involving the performance of minimally invasive surgery point to a growing body of evidence which supports the concept of delivering an irrigant to the port site after the surgical procedure. In these studies, the irrigants were delivered by a syringe and needle and included substances such as betadine, saline and lidocaine. These studies showed that irrigating the port site with such substances immediately after the surgical procedure beneficially resulted in a lower incidence of infection or less pain, depending on the irrigant. The technique, however, also resulted in increased operative time and increased exposure of the surgical staff to needle sticks. In addition, the potential for contaminants to spread to the port site during the surgery has been well documented. Irrigation performed only at the end of the surgical procedure unfortunately cannot reduce patient exposure to contaminants during the procedure.

In addition, in laparoscopic, arthroscopic, and other minimally invasive procedures, there can be four basic stages of pain initiators: incision, trocar insertion, trocar manipulation, and trocar removal. These stages result in the exacerbation of the pain cascade. The pain cycle begins when there is local injury to the surrounding tissues which is sensed by the nociceptors that send signals to the spinal cord and onto the brain. The brain then sends additional signals back to the local site of damage. The patient, who is under general anesthesia, is not able to withdraw from the painful stimulus.

The neurotransmitters which communicate in the language of pain are released continuously. These neurotransmitters are some of the factors responsible for the post operative pain that the patient feels. As these substances affect the local area of damage, they result in a predictable pain legacy which is felt by the patient for hours and days after the time of injury.

To lower the release of the neurotransmitters and subsequent sensitivity to innocuous irritation, lower pain threshold, and decrease excitability associated with the pain cascade, injections of a local anesthetic are made to the ports site during the surgery. There are, however, several problems with this technique. First a needle and syringe are needed to deliver the local anesthetic to the port site. Second, the injection is in an extrapolated direction of where the obliquely inserted trocar's path lies and, therefore, the anesthetic may not be accurately delivered to the trocar damaged area. This results in a time dependent diffusion and less than 360 degree infiltration of the anesthetic to the location of the damaged nociceptors and allows a variety of internally released biological substances and receptors to propagate the pain cascade. Delivery of the local anesthetic by a syringe and needle also adds to the number of surgical steps to be performed and increases the chances of needle sticks. In addition, the pain medications delivered by a syringe and needle have been short acting local anesthetics and given only once. As a result of the necessity to deliver the medication via the needle and syringe, and the fact that the medication is short acting in its nature, the patient frequently experiences “break through pain” at the port site. This results in more pain medication delivered to the post operative laparoscopic patient. Literature has shown that when pain medication is given multiple times to the port site, there is additional benefit to the patient in terms of less pain in the recovery room, i.e., less “break through pain” at the port site. This concept is known as “summation.” Thus there is a need for a device which will enable the surgeon to deliver the medication multiple times throughout the case without a needle and syringe.

In view of the above-mentioned drawbacks in the field, there is a need for more effective and accurate delivery of biologically active agents to an access point or port in the body of a patient and to the intervening layers of the skin, such as the epidermis and dermis and associated subcutaneous tissue, before, during, and/or after the performance of minimally invasive surgery. Such delivery of active agents could result in patient benefits, such as through the delivery of anesthetics, cancer treatment medication or other pharmaceutical compounds, as well as reduction of port site contamination and infection, and reduction of post-operative pain. Other uses of the invention may be made in connection with delivering any desired fluid and pharmaceutically acceptable formulations that contain a biologically active agent to a patient.

### SUMMARY OF THE INVENTION

The present invention generally relates to a unique fluid delivery cannula which provides an interface between an access point or port in the body of a patient and a working channel which may receive tools or instruments used during minimally invasive surgery. In accordance with one general aspect of the invention, the cannula allows introduction of at least one biologically active agent in a pharmaceutically acceptable formulation, or composition, at the port site, or another site within the body of the patient, at any time after the cannula is introduced through the access point or port site of the body. The agent in the formulation may be present in a freely soluble form for an immediate effect or entrapped in a biologically degradable matrix for a sustained effect, or both. The agents may be introduced manually, such as through a manually operated syringe coupled in communication with one or more delivery passages in or on the wall of the cannula. Alternatively, the agents may be delivered automatically through a suitable medical pump or other device. The agents may include, for example, saline solution, local anesthetic compounds, betadine-containing fluids, or other biologically active agents or substances in a pharmaceutically acceptable formulation, depending on the intended use and desired purpose. In various manners, the cannula may also deliver a biologically degradable matrix that entraps at least one active agent which is released from the matrix in a time controlled manner as the matrix degrades. Presently, it is contemplated that such agents will be especially beneficial to reduce post-operative pain, prevent infection and contamination at the port site and provide for many types of treatment to an affected area within the body of the patient.

In one embodiment, the delivery cannula releasably attaches to the hub. In another embodiment, the delivery cannula is integrally formed with at least a portion of the hub. As one example, the delivery cannula may be integrally molded with a housing portion which is configured to receive valving components and/or other insufflation components, while also allowing the trocar to pass through into the delivery cannula. The hub can include a fluid inlet comprising a coupling, such as a standard luer connection, for receiving a manually operated syringe device allowing for the injection of the desired active agents. The delivery cannula preferably has, in addition to a main lumen for receiving the trocar, one or more passages for purposes of delivering one or more biologically active agents to the port site.

Instead of or, or in addition to the fluid delivery passages discussed above, the cannula itself can carry one or more biologically active agents. For example, the outer surface of the cannula can carry a biologically degradable matrix that contains at least one biologically active agent such as a long term pain medication, a short term medication, or both. Such medication(s) might be carried in various forms on the outside surface of the cannula, or molded into the cannula in a polymeric matrix. Since it can be important to immediately deliver active pain medication to the tissue surrounding the port site, the short term pain medication may be delivered via a pump or syringe device and the long term pain medication may degrade from the outer surface of the cannula during the surgical procedure. In this manner, the long term pain medication is absorbed by the surrounding tissue for effecting long term pain relief for one or more days following surgery. Another option is to first inject the short term pain medication through a syringe or pump and then inject the long term pain medication again through a syringe or pump device, or via another injection method using the delivery passages of the cannula.

In one embodiment, the cannula has a layered construction with multiple passages contained between two layers of the cannula. The outside surface of one layer of the cannula includes grooves or recesses in communication with the inlet and an outer layer of the cannula includes one or more apertures or perforations communicating with the grooves for dispensing the active agent. Also in the embodiment, the outside portion of the cannula, which has the active agent dispensing apertures, provides a visual target zone for the accurate delivery of the active agent to the port site. For example, this may comprise using a different color, texture, or other visually identifiable indicia at the active agent dispensing location of the cannula such that the surgeon can accurately determine where the active agent is being directed. Biologically active agents may alternatively only be incorporated into or onto the cannula such that they degrade or other wise leach into the tissue while the cannula is in place in a patient.

The invention may be manufactured in many different manners while still functioning in accordance with the inventive principles. As mentioned above, an embodiment of the invention includes an inner cannula member having a grooved outer surface to define multiple fluid passages. An outer layer of biocompatible material (e.g., PTFE) is preferably heat shrunk onto the outer surface to enclose and seal the grooves to form passages. The outer layer may be coated with or otherwise carry a biodegradable matrix that contains at least one biologically active agent that is time released. The biocompatible material includes several apertures positioned around the circumference of the cannula and communicating with the grooves so that the fluid may be dispensed around the entire circumference of the cannula at a specific location along the length thereof. Alternatively, or in addition, fluid passages and one or more apertures may be provided only at one location about the circumference of the cannula for even more targeted delivery of the fluid.

As alternative embodiments, the outer layer may be comprised of a biodegradable component that contains an active agent which is time released. The outer layer may be configured similar to a condom and rolled onto the cannula and which includes the necessary aperture(s) and/or channels for delivery of the active agent to the patient from a medical pump or syringe. The outer layer may be a rigid layer which is coupled to the inner cannula member in a rigid fashion or, for example, in a movable fashion such as a rotatable fashion to allow opening, closing, or size adjustment of the delivery passage(s). As one additional alternative, the outer layer may be formed at least partially of a porous material which provides the necessary apertures. Such porous materials may, for example, take the form of sintered metals, filter media, paper, mesh cloth or a porous plastic. The porous material may also be formed of or contain a biodegradable matrix that entraps at least one biologically active agent. As the matrix degrades, the agent is released in a controlled manner resulting in a sustained effect of the agent over a period of time.

Another embodiment of the invention provides an expandable sleeve that may itself comprise a cannula through which a trocar or trocar assembly is inserted or which may take the place of the perforated outer layer of the grooved cannula discussed above. Other expandable sleeve embodiments may also be configured in accordance with this aspect of the invention as well. Such an expandable sleeve can, for example, allow trocars having different diameters to be inserted through the sleeve. Therefore, the same expandable fluid delivery sleeve may be used in connection with different sized trocars or trocar assemblies thereby reducing or eliminating the need for different sized fluid delivery cannulas or sleeves. The sleeve may also be associated with a biodegradable matrix that contains one or more biologically active agents that are time released as the matrix degrades.

Various objects, advantages and features of the invention will become more readily apparent to those of ordinary skill upon review of the following detailed description of the preferred embodiment taken in conjunction with the accompanying drawings.

### BRIEF DESCRIPTION OF THE DRAWINGS

FIG. 1 is a perspective view showing a trocar-fluid delivery cannula complex constructed in accordance with the invention and being used during a minimally invasive surgical procedure.

FIG. 2 is a cross sectional view taken generally along the longitudinal axis of the trocar-fluid delivery cannula complex of FIG. 1 for showing the irrigant flow path.

FIG. 3 is an enlarged cross sectional view similar to FIG. 2, but more clearly showing the flow path for the delivery of fluid through the cannula.

FIG. 4 is a cross sectional view taken along line 4-4 of FIG. 2.

FIG. 5 is a plan view of the fluid delivery cannula with the outer layer or sheath removed for clarity.

FIG. 6 is a plan view of another embodiment in which the fluid delivery cannula is integrally formed with a portion of a trocar hub.

FIG. 7 is a cross sectional view taken along line 7-7 of FIG. 6.

FIG. 8 is a longitudinal cross sectional view similar to FIG. 2, but illustrating an alternative embodiment of the invention incorporating an expandable fluid delivery sleeve.

FIG. 9 is a perspective view of another alternative embodiment of an expandable fluid delivery sleeve or cannula.

FIG. 10 is a cross sectional view taken along line 10-10 of FIG. 9.

FIG. 11 is an enlarged perspective view of the distal end of another expandable fluid delivery sleeve or cannula.

### DETAILED DESCRIPTION OF THE PREFERRED EMBODIMENTS

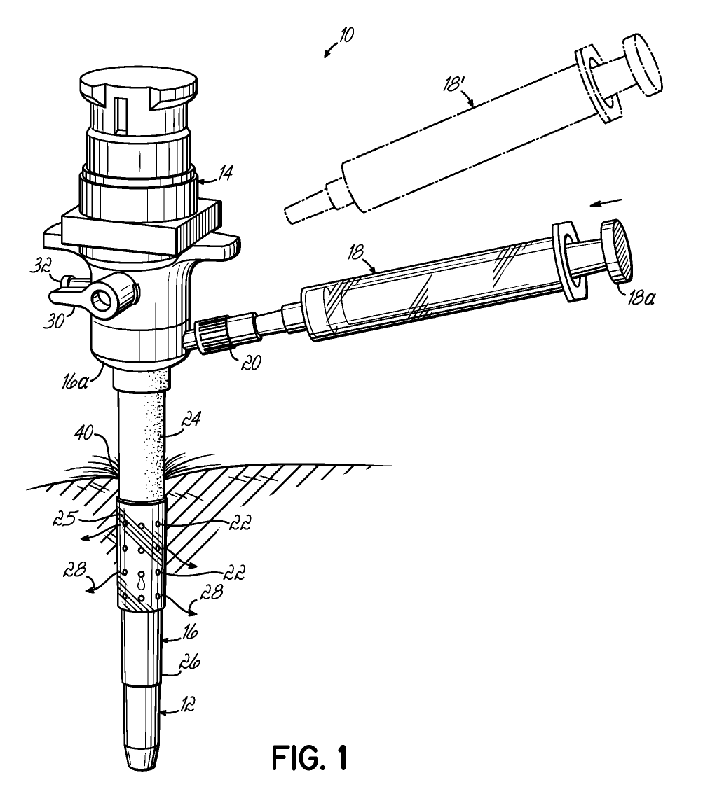

FIG. 1 illustrates a trocar-fluid delivery cannula complex 10 constructed in accordance with one preferred embodiment of the invention. Complex 10 includes a trocar assembly 12 which may include a conventional hub assembly 14. Representative trocar assemblies are shown and described in previous patents, such as my previous U.S. Pat. Nos. 6,063,060; 6,039,725; 5,865,817; and 5,865,809, and PCT Application No. PCT/US02/29356, the disclosures of which are hereby fully incorporated by reference herein. The present invention may implemented into cannulas associated with various other minimally invasive procedures including, but not limited to, laparoscopic procedures.

In accordance with the invention, a cannula 16 is positioned on the outside of trocar assembly 12 and includes a base portion 16a. A syringe 18 couples to base portion 16a of cannula 16 through a fluid coupling, such as a standard luer connector assembly 20. A second syringe 18′ may be provided to, for example, supply a second biologically active agent. In this regard, syringe 18 may be used to first supply a short term or immediately active pain medication whereas syringe 18′ may be used to supply a long term or time release pain medication. A plunger 18a of syringe 18 is used to manually inject a fluid into base portion 16a of cannula 16. An outer layer or sheath 24, is secured to the outer surface of an inner tube 26 of cannula 16 and includes apertures 22. Another layer 25, which includes a biologically active agent, may be adhered to sheath 24 or otherwise incorporated into or onto cannula 16, depending on the application needs. The sheath 24 itself or any other portion of the cannula 16 which contacts the damaged tissue of the patient could alternatively be formed from or otherwise carry a material which incorporates a biologically active agent for delivery to the patient.

In one preferred embodiment, sheath 24 is a tube which is heat shrunk onto inner tube 26 but it may take other forms and may be secured in other ways. As will be described below, cannula 16 includes appropriate fluid passages communicating with an inlet passage in base portion 16a to allow the fluid to be dispensed through apertures 22 as shown by arrows 28. If necessary, hub assembly 14 an further include an insufflation valve 30 and a gas inlet 32 for receiving a pressurized gas, such as CO₂.

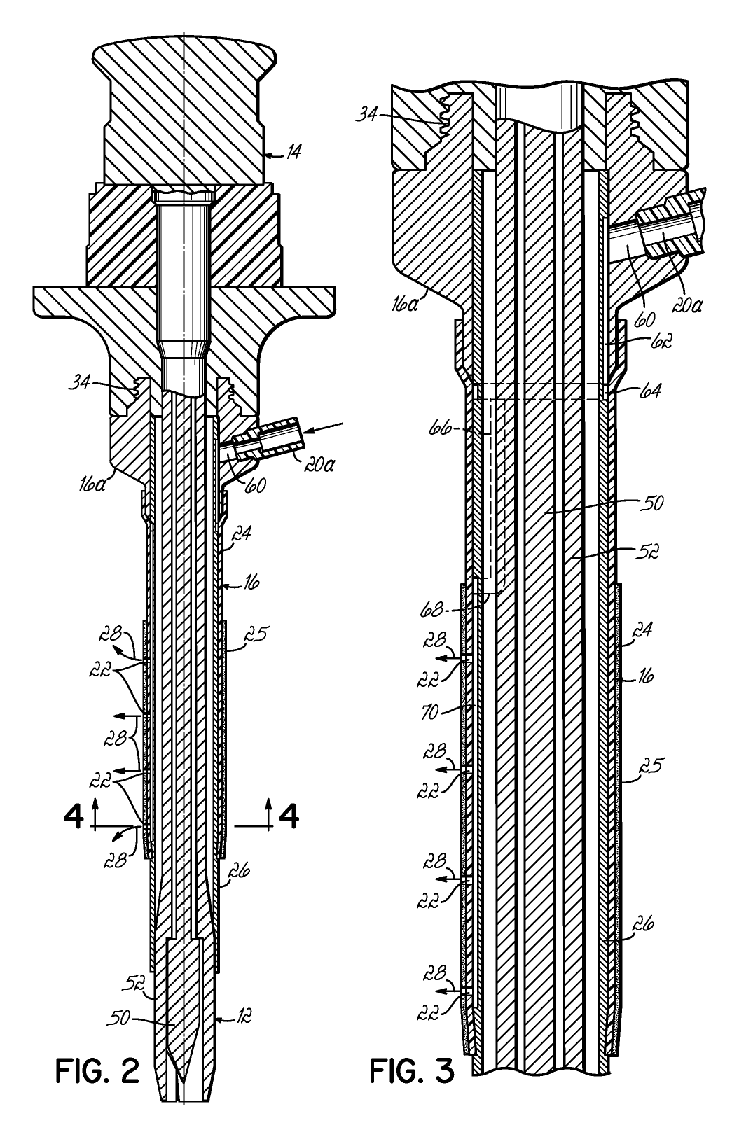

As further shown in FIGS. 2 and 3, base portion 16a of cannula 16 is threaded onto hub assembly 14 by threads 34. Thus, cannula 16 may be easily coupled to and decoupled from hub assembly 14. In the preferred embodiment, cannula 16 is disposable, however, it also may be manufactured as a reusable device intended to be sterilized between uses. Trocar assembly 12 more specifically comprises a trocar 50 received by a protective shield 52. It will be appreciated that other instruments and tools may be inserted through the working channels formed by either irrigating cannula 16 or other tubular member(s) positioned within cannula 16. This includes many other configurations of trocars or trocar assemblies as generally recognized in the art.

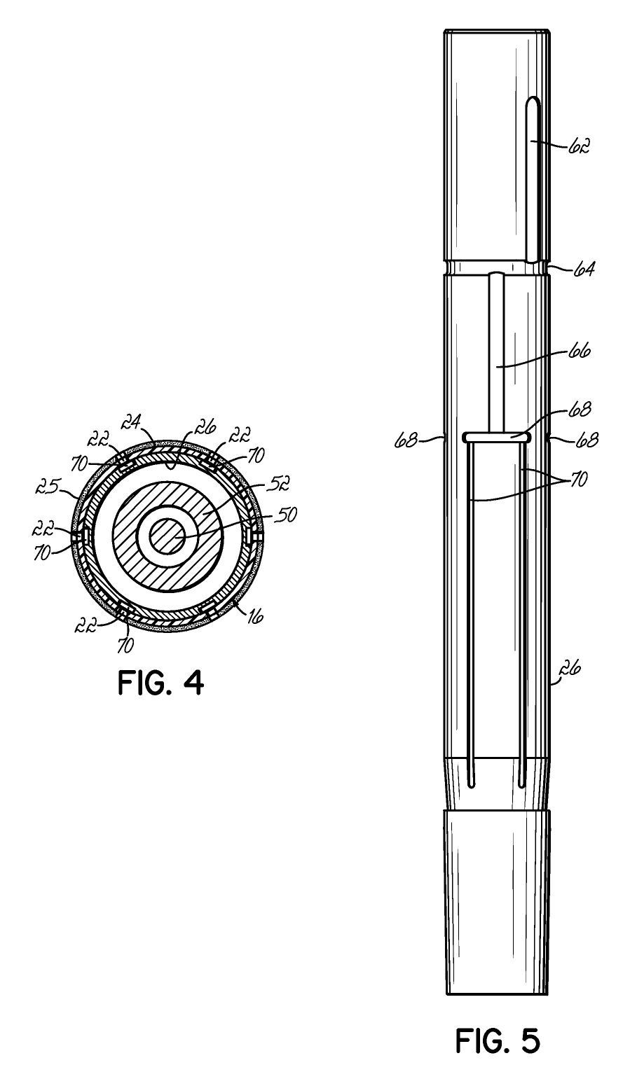

More specifically referring to FIGS. 3-5, irrigation fluids and the active agents in a pharmaceutically acceptable formulation are introduced through luer connector 20a (FIG. 3) into fluid inlet 60 and groove or channel 62 formed in inner tube 26 of cannula 16. Groove 62 communicates with an annular, circumferential groove 64 and groove 64 communicates with three separate longitudinal grooves 66 which are spaced in 1201 increments about inner tube 26. Grooves 66 respectively communicate with three partially annular grooves 68 which, in turn, each communicate with two longitudinal grooves 70. Longitudinal grooves 70 communicate with apertures 22 in sheath 24 and apertures 22 thereby dispense the fluid at the port site 40 or, if cannula 16 is appropriately inserted and positioned, elsewhere within the patient.

As mentioned above, the outer sheath 24 of the cannula 16 may be formed of PTFE and may be transparent or at least translucent. In addition, the area of sheath 24 containing apertures 22 may be formed with a distinct color, texture or other visually identifiable indicia which allows the surgeon to accurately position the apertures 22 with respect to the tissue to be infused with an irrigation fluid or active agent in a pharmaceutically acceptable formulation. The various grooves in the outside surface of the inner tube 26 may be substituted with one or more passages within the walls of the inner tube 26 and may be of any suitable configuration and shape so long as the function of delivering the fluid or formulation through the wall of the cannula 16 is facilitated by the configuration. The outer wall or sheath is a heat shrinkable material, such as an elastomeric material, however, this may also be substituted by other components or even eliminated, for example, if the passages and apertures are in the wall of an integrally formed cannula or if another fluid or active agent delivery structure is carried on the outer cannula. The inner tube in the embodiment is formed from aluminum with the various grooves in its outer surface being machined, however, it may instead be formed of other materials, such as plastic materials, and formed by other techniques such as molding. The preferred embodiment is especially advantageous in that it is simple to manufacture and the outer sheath forms a seal at the upper and lower ends of the inner tube while, at the same time, defining walls of the internal passages formed by the various grooves.

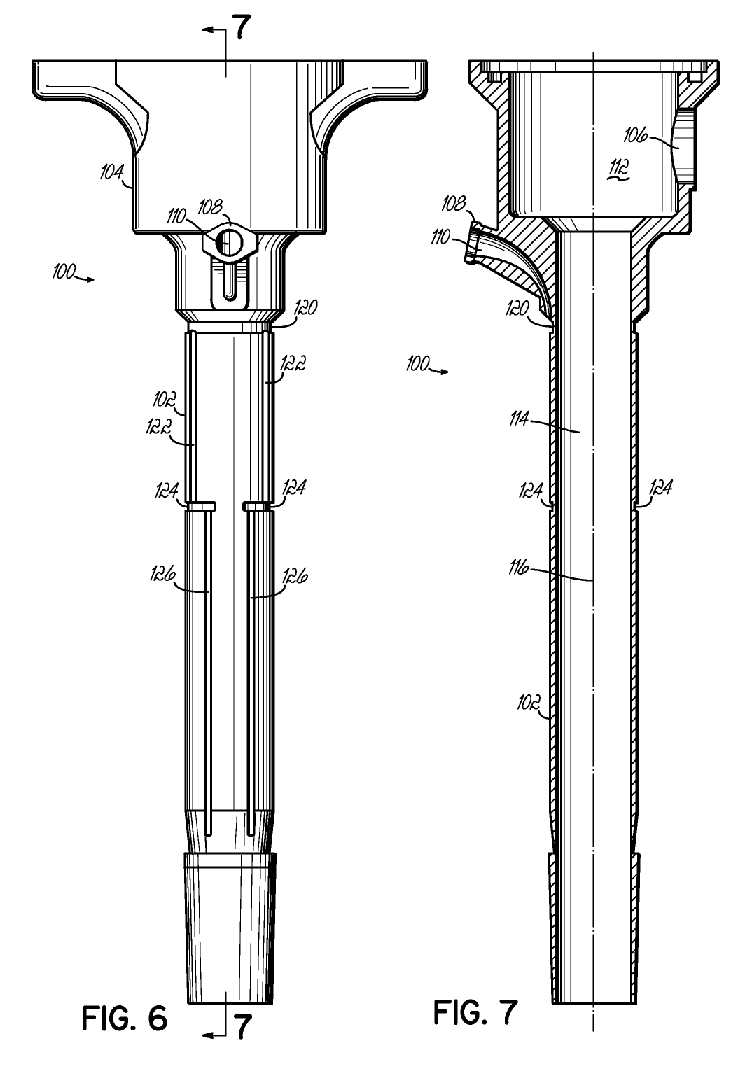

FIGS. 6 and 7 illustrate a second illustrative embodiment of the invention comprising a delivery cannula 100 which includes an irrigating portion 102 and a hub or housing portion 104 formed in one piece. For example, the entire structure shown in FIGS. 6 and 7 may be molded from a polymeric material, such as conventional medical grade polymers, using Mu-cell technology or other appropriate molding techniques. In FIGS. 6 and 7, the outer layer or sheath containing the one or more perforations has been removed for clarity. Housing portion 104 includes a port 106 for receiving valving and gas input components as are known in the art. A fluid input 108 is formed on cannula 100 and communicates with a passage 110 for the introduction of the necessary or desired fluids to irrigation portion 102. A space 112 is provided for the necessary valving, sealing components, etc., typically used in trocar hubs. A lumen 114 extends along an axis 116 for receiving the trocar (not shown) and other working instruments. A system of fluid delivery passages is formed on the outside surface of irrigation portion 102 in the same illustrative pattern as discussed with respect to the first embodiment. This includes an annular groove 120 which communicates with passage 110 and delivers the fluid to three separate longitudinal passages 122 positioned at 1201 increments around the outside surface of irrigation portion 102 relative to axis 116. Grooves 122 communicate with respective partially annular grooves 124. Again, while only two grooves 124 are shown in the drawings, a total of three grooves are formed in the outer surface of irrigation portion 102 positioned at 1201 increments about axis 116. Each partially annular groove 124 communicates with two separate longitudinal grooves 126. Although only two grooves 126 are shown in FIG. 6, it will be appreciated that a total of six such grooves are formed in the outer surface of irrigation portion 102 in this particular embodiment. As in the first embodiment, grooves 126 communicate the fluid to perforations in the outer sheath (not shown) which then deliver the fluid to the patient. The outer sheath, as in the first embodiment, is heat shrunk onto irrigation portion 102 so as to seal all of the grooves in the same manner as shown, for example, in FIGS. 2 and 3 of the first embodiment. Alternatively, a film that contains one or more controlled release active agents in a biologically degradable matrix may also be used. As mentioned above, it will be appreciated that many other configurations of fluid delivery passages may be utilized in the cannula within the spirit and scope of this invention.

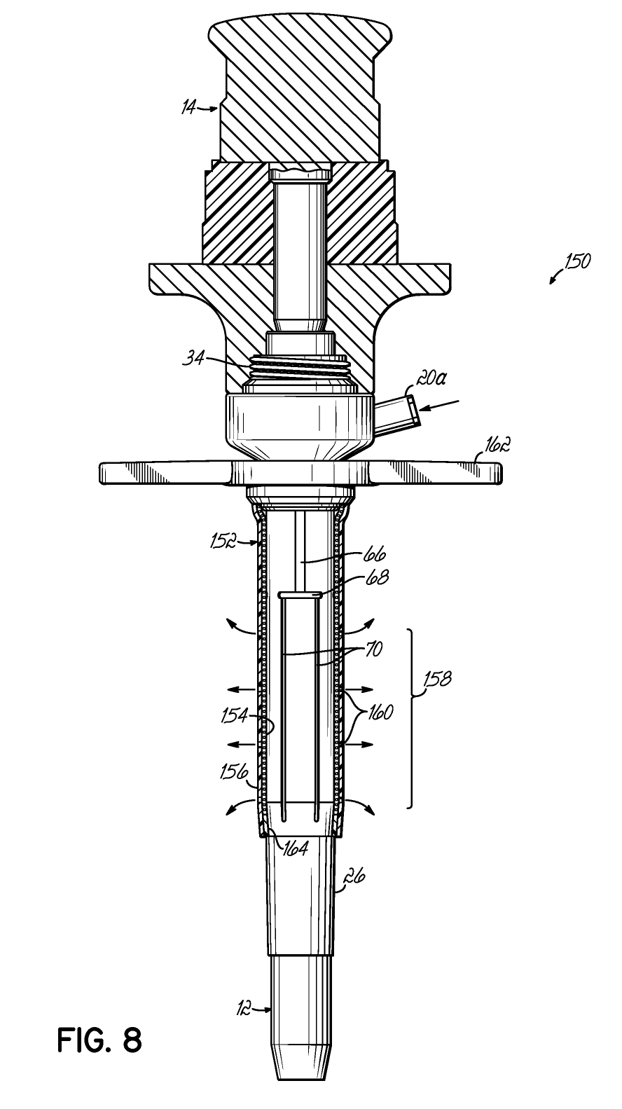

In FIG. 8, like reference numerals refer to like elements of structure between the two embodiments. In the alternative trocar-cannula complex 150 of FIG. 8, the outer sleeve or layer 24 (not shown) which was affixed to the grooved cannula 26 has been removed and replaced by an expandable sleeve 152. Expandable sleeve 152 may be a layered construction including a mesh layer 154 and an outer elastomeric layer 156. Layer 156 is uniformly perforated about its entire periphery, such as in a circumferential zone 158 as shown in FIG. 8, so that at least some of the perforations 160 line up with the longitudinal grooves 70 of the cannula 26. Thus, fluid is delivered through input 20a and into grooves 66, 68, 70 as described previously with respect to the first embodiment and this fluid is transferred through the expandable inner mesh layer 154 and expandable outer elastomeric layer 156 containing perforations 160. It will be appreciated that many other forms than the layered mesh construction shown may be used in place of the expandable sleeve 152 shown in FIG. 8. FIG. 8 illustrates the use of the expandable sleeve 152 in connection with a 10 mm trocar assembly, however, in accordance with this aspect of the invention, the expandable fluid delivery sleeve 152 may alternatively be used with other trocars having larger or smaller diameters. A rigid handle portion 162 is provided at the proximal end of sleeve 152 to allow application and removal of sleeve 152 to and from trocar 12. In order to seal the distal end of the expandable sleeve, a seal 164 may be provided distally of the mesh layer 154 as generally illustrated in FIG. 8. Alternatively, this seal 164 may be eliminated and the mesh layer 154 could then allow additional fluid to be delivered from the distal end of the sleeve 152.

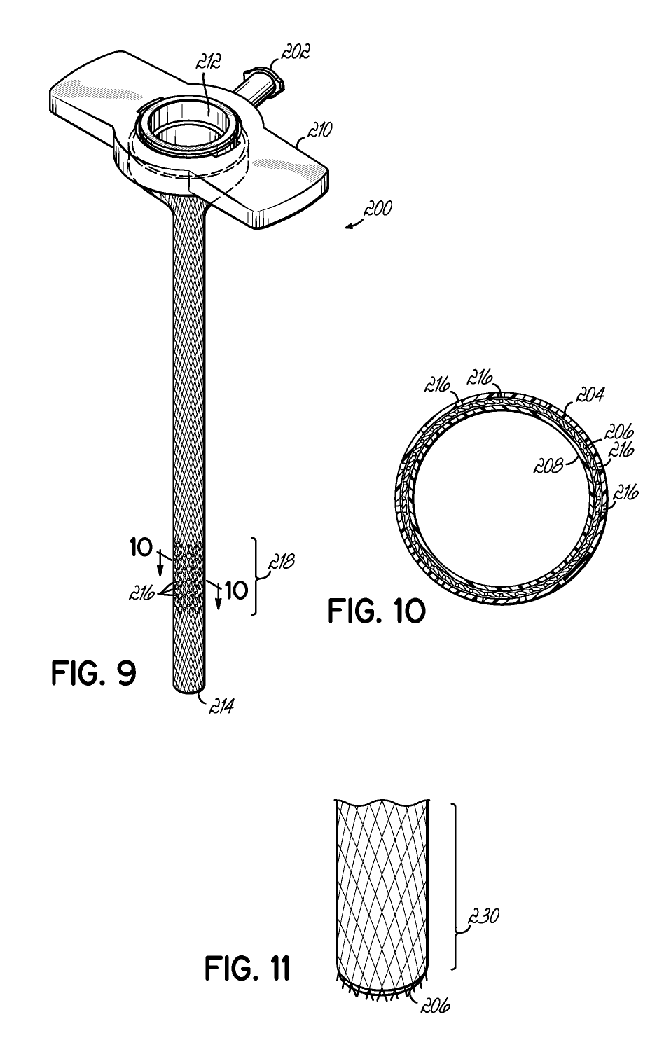

FIGS. 9 and 10 illustrate another embodiment of an expandable fluid delivery sleeve 200 which does not need the separate cannula 26 (FIG. 8) for fluid delivery as in the embodiment of FIG. 8. Instead, this sleeve 200 is formed in a manner allowing fluid delivery to take place via an input 202 and sleeve 200 alone. Sleeve 200 is formed of a layered construction including an outer perforated layer 204, an intermediate mesh layer 206, and an inner layer 208. These layers may be coated with a biologically degradable matrix that contains an active agent. Each layer 204, 206, 208 is expandable such that sleeve 200 may be used effectively on trocars having different diameters. The intermediate mesh layer 206 allows fluid to travel through the interstices therein from an appropriate delivery passageway extending through input 202 and an upper handle portion 210. Alternatively, other types of delivery passages may be utilized. A trocar (not shown) is inserted through the bore 212 at the proximal end such that it extends through the distal end 214 of the expandable sleeve 200. Perforations 216 are preferably formed in a desired zone 218 of sleeve 200 generally as described with respect to the previous embodiments. This zone 218 may be formed of a different color or in any other manner which indicates the positioning of the perforations to the doctor during the surgical procedure. Although not shown in FIGS. 9 and 10, this sleeve 200 may also have a seal at the distal end 214 to prevent fluid from leaking out the distal end 214.

As exemplified in FIG. 11, a distal end 230 of the expandable sleeves may be formed so as to allow fluid delivery to take place directly at the distal end. This aspect is shown in FIG. 11 schematically by indicating that the intermediate mesh layer 206 extends slightly beyond the other layers or is otherwise unsealed and, therefore, the fluid pathway through the mesh material 206 remains unblocked at the distal end 230. This general aspect of fluid delivery from the distal end 230 may be used alone or in conjunction with fluid delivery from surface perforations as previously described.

Many different types of irrigation fluids may be introduced through the fluid delivery cannulas of this invention. These include, but are not limited to, saline solutions, lidocaine-containing fluids, betadine-containing fluids, cancer treatment fluids, or any other fluid or pharmaceutically acceptable formulation necessary or desired for a particular medical procedure. In addition, fluids other than irrigation fluids or treatment fluids may be delivered through the cannulas of this invention. As one additional example, bioadhesives may be delivered to an incision site or any other necessary tissue repair site to provide for quicker and more effective administration of the adhesive to the desired site.

These fluids are pharmaceutically acceptable formulations that contain biologically active agents that the surgeon can infuse to the port site and intervening tissue layers. Examples of active agents include, but are not limited to various types of anesthetics, therapeutic polypeptides, steroids, antiangiogenic agents, cancer chemotherapeutic agents, anti-infectives (antibiotics, antiviral, etc.), cytotoxins, anticoagulants, fibrinolytic agents, anti-inflammatory agents and combinations thereof.

The pharmaceutically acceptable formulations as known to one skilled in the art may contain the biologically active agents in a freely soluble form for immediate effect at the tissue site or in a controlled or sustained release matrix for a long-term effect such as hours or days, or a combination of both.

The controlled or sustained released matrix may be biologically degradable and prepared using procedures as known to one skilled in the art. The form of the matrix may be selected from microporous films, microspheres, nanospheres, micelles, liposomes, powders, microparticles, and hydrogels. These matrices may be a component of the pharmaceutically acceptable formulation that is delivered to the port site by a syringe or pump. They then diffuse into the surrounding tissue and become embedded or implanted in the tissue. Thus, they impart a sustained effect of the active agent due to its controlled release from the matrix as it degrades.

In addition, the biologically degradable matrices may also be carried on the outside of the cannula, such as in a microporous film or other form. Alternatively, the matrices may be carried on the surface of cannula as microparticles, nanospheres, powders, hydrogels, etc. The matrix may be a component of the outer surface or layer of the cannula or a disposable cannula. The cannula may alternatively be molded from a polymer matrix comprised of the desired biologically active agent. The disposable cannula, with a coating of the matrix, may be provided in a kit or packaged form.

Biologically degradable matrices may be formed by procedures known to one skilled in the art. For example, such components may be various types of lipids that form micelles and liposomes, polymers and copolymers of polyorthoesters, polyethylene glycol, ketene acetals, polyols and others. Examples of the various biodegradable polymers, various biologically active agents that become entrapped or encapsulated in the formed matrices as previously described, injectable fluid dosage forms, and semi-solid pharmaceutical compositions are described in U.S. Pat. No. 6,524,606; U.S. Pat. No. 6,667,371; U.S. Pat. No. 6,613,355; U.S. Pat. No. 5,968,543; U.S. Pat. No. 5,939,453; U.S. Pat. No. 4,957,998; U.S. Pat. No. 4,946,931; U.S. Pat. No. 4,855,132; U.S. Pat. No. 4,764,364; U.S. Pat. No. 4,304,767; and U.S. Published Applications 2002/0037300, 2003/0130472, 2002/0168336, 2002/0176844, and 2003/0212148, the disclosures of which are incorporated herein in their entirety. Other dosage forms and biologically active agents in pharmaceutically acceptable formulations may be used as well.

Many different types of trocars and cannulas may be utilized within the scope of this invention. These trocars and cannulas may be inserted through a port site of a patient together in one operation or separately, for example, by using a needle introducer for an expandable cannula and subsequently introducing the trocar.

The use of the invention eliminates or at least reduces the handling of needles during the surgical procedure and the trocar cannula assembly allows accurate delivery to the port site. The active agent is delivered to the port site fast and simple and in a 360 degree fashion. Both short and long acting active agents may be delivered to ameliorate various biological responses such as the pain cascade in a physiological fashion. The assembly also allows the surgeon to choose what to infuse or irrigate for any particular case and may be infused at any time during the procedure and as many times as is necessary such as after the initial introduction of the assembly through the port site, during the surgical procedure, or at the end of the procedure.

While the present invention has been illustrated by a description of a preferred embodiment and while this embodiment has been described in some detail, it is not the intention of the Applicant to restrict or in any way limit the scope of the appended claims to such detail. Additional advantages and modifications will readily appear to those skilled in the art. The various features of the invention may be used alone or in numerous combinations depending on the needs and preferences of the user. This has been a description of the present invention, along with the preferred methods of practicing the present invention as currently known. However, the invention itself should only be defined by the appended claims, wherein I claim:

1. A trocar-cannula complex for use in minimally invasive surgical procedures performed through a port site of a patient and in the delivery of biologically active agents to the patient comprising:
a trocar; and
a cannula comprising a tubular structure including a central lumen receiving the trocar and an outer surface adapted to interface with tissue at the port site; a first delivery mechanism associated with the cannula for delivering a first biologically active agent to a patient; and a second delivery mechanism for delivery of a second biologically active agent to the patient.

2. The trocar-cannula complex of claim 1, further comprising a hub portion having valving components operative to deliver insufflation gas to the patient, the hub portion being coupled to the cannula in a releasable manner.

3. The trocar-cannula complex of claim 1, further comprising a hub portion having valving components operative to deliver insufflation gas to the patient, the hub portion being formed integrally with the cannula.

4. The trocar-cannula complex of claim 3, wherein the hub portion and the cannula are integrally molded from a polymeric material.

5. The trocar-cannula complex of claim 1, wherein said tubular structure is radially expandable.

6. The trocar-cannula complex of claim 1, wherein the first delivery mechanism is a biologically degradable matrix containing the first biologically active agent and is associated with the cannula.

7. The trocar-cannula complex of claim 6, wherein the matrix is selected from the group consisting of microporous films, microspheres, nanospheres, micelles, powders, microparticles, hydrogels, and combinations thereof.

8. The trocar-cannula complex of claim 7, wherein the microporous films have channels.

9. The trocar-cannula complex of claim 1, wherein the second delivery mechanism is selected from the group consisting of pumps, syringes, and combinations thereof.

10. The trocar-cannula complex of claim 2, wherein the hub is associated with the second delivery mechanism.

11. The trocar-cannula complex of claim 8, wherein the channels communicate with the second delivery mechanism.

12. A trocar-cannula complex for use in minimally invasive surgical procedures performed through a port site of a patient and delivery of biologically active agents to a patient comprising:
a trocar; and
a cannula comprising a multilayer tubular structure including a central lumen receiving the trocar and an outer surface associated with a first delivery mechanism for delivering a first biologically active agent and adapted to interface with tissue at the port site, the cannula further including at least one delivery passage having an inlet and an outlet and being at least partially positioned between two separate layers of the tubular structure, the outlet communicating with the outer surface for delivering a second biologically active agent through the passage from a second delivery mechanism.

13. The trocar-cannula complex of claim 12, wherein the two separate layers include an inner rigid tubular member and an outer sheath carried on the inner rigid tubular member, the inner rigid tubular member including a grooved surface for providing the passage and the outer sheath operative to seal the passage against leakage.

14. The trocar-cannula complex of claim 13, wherein said outer sheath is comprised of a polymeric material carried on the grooved surface.

15. The trocar-cannula complex of claim 14, wherein the polymeric material includes PTFE and is associated with a biologically degradable matrix.

16. The trocar-cannula complex of claim 14, wherein said outer sheath is heat shrunk onto said grooved outer surface.

17. The trocar-cannula complex of claim 13, wherein said outer sheath is radially expandable.

18. The trocar-cannula complex of claim 12, wherein the first delivery mechanism is a biologically degradable matrix and is associated with the outer sheath.

19. The trocar-cannula complex of claim 12, wherein the second delivery mechanism is selected from the group consisting of pumps, syringes, and combinations thereof.

20. A cannula for use in minimally invasive surgical procedures and delivery of biologically active agents performed through a port site of a patient comprising:
a tubular structure including a central lumen configured to receive a trocar and an outer surface adapted to interface with tissue at the port site and associated with a first delivery mechanism for the delivery of a first biologically active agent, the tubular structure further including at least one delivery passage having an inlet and an outlet, the outlet communicating with the outer surface for delivering a second biologically active agent thereto from a second delivery mechanism.

21. The cannula of claim 20, wherein the tubular structure is formed by multiple layers and the delivery passage is located between at least two of the layers.

22. The cannula of claim 20, further comprising a hub portion having valving components operative to deliver insufflation gas to the patient, the hub portion being coupled to the tubular structure in a releasable manner and associated with the second delivery mechanism.

23. The cannula of claim 20, further comprising a hub portion having valving components operative to deliver insufflation gas to the patient, the hub portion being formed integrally with the tubular structure and associated with the second delivery mechanism.

24. The cannula of claim 23, wherein the hub portion of the tubular structure are integrally molded from a polymeric material.

25. The cannula of claim 20, wherein tubular structure further comprises at least two separate layers include an inner rigid tubular member and an outer sheath carried on the inner rigid tubular member, the inner rigid tubular member including a grooved surface for providing the delivery passage and the outer sheath operative to seal the delivery passage against leakage.

26. The cannula of claim 25, wherein the outer sheath is comprised of a polymeric material carried on the grooved surface.

27. The cannula of claim 25, wherein the polymeric material includes PTFE.

28. The cannula of claim 26, wherein the outer sheath is heat shrunk onto the grooved outer surface.

29. The cannula of claim 28, wherein the sheath is associated with the first delivery mechanism.

30. The cannula of claim 20, wherein the tubular structure is radially expandable.

31. The cannula of claim 19, wherein the first delivery mechanism is a biologically degradable matrix.

32. The cannula of claim 19, wherein the second delivery mechanism is selected from the group consisting of pumps, syringes and combinations thereof.

33. A cannula for use in minimally invasive surgical procedures performed through a port site of a patient and delivery of biologically active agents to the patient comprising:
a radially expandable tubular structure including a central lumen configured to receive a trocar and an outer surface adapted to interface with tissue at the port site and associated with a first delivery mechanism for delivery of a first biologically active agent, the tubular structure further including a distal end and at least one delivery passage having an inlet and an outlet, the outlet communicating with at least one of the outer surface and the distal end for delivering a second biologically active agent from a second delivery mechanism.

34. A method for dispensing biologically active agents from a first delivery mechanism and a second delivery mechanism associated with a trocar-cannula assembly having a proximal end, a distal end, a plurality of delivery channels and an exterior surface extending between the proximal and distal ends and communicating with the delivery channel comprising:
introducing the trocar-cannula assembly into a body cavity through a port site of a patient contacting a surface of the wall of the port site;
delivering a first biologically active agent from the first delivery mechanism and a second biologically active agent from the second delivery mechanism to the surface of the walls and inner and outer surfaces of the port site;
removing the trocar from the assembly;
advancing a gas into the body cavity;
performing a surgical procedure; and
removing the cannula from the patient upon completion of the surgical procedure.

35. The method of claim 34, wherein the first delivery mechanism is a biologically degradable matrix for sustained release of biologically active agents.

36. The method of claim 35, wherein the matrix is in a form selected from the group consisting of microporous films, microspheres, nanospheres, micelles, liposomes, powders, microparticles, hydrogels, component of an outer surface of the cannula, and combinations thereof.

37. The method of claim 34, wherein the second delivery mechanism is selected from the group consisting of pumps, syringes, and combinations thereof.

38. The method of claim 34, wherein the first active agent and second active agent are an anesthetic either or in combination with another active agent selected from the group consisting of anesthetics, therapeutic polypeptides, steroids, antiangiogenic agents, cancer chemotherapeutic agents, anti-infectives, cytotoxins, anticoagulants, fibrinolytic agents, anti-inflammatory agents and combinations thereof.

39. The method of claim 34, wherein the second biologically active agent is in a pharmaceutically acceptable formulation.

40. The method of claim 39, wherein the pharmaceutically active formulation contains the biologically active agent in a form selected from the group consisting of a freely soluble form for immediate effect, entrapped in a biologically degradable matrix for a sustained release and effect, and a combination of both.

41. The trocar-cannula complex of claim 3, wherein the hub is associated with the second delivery mechanism.
